(19) United States
(12) Patent Application Publication
(10) Pub. No.: US 2019/0093060 A1
(43) Pub. Date: Mar. 28, 2019

(54) CELL CULTURE BASE MATERIAL FOR TRAIT INDUCTION CONTROL OF MESENCHYMAL STEM CELLS AND TRAIT CONTROL METHOD THEREOF

(71) Applicant: TOKYO OHKA KOGYO CO., LTD., Kawasaki-shi (JP)

(72) Inventors: Emi MAENO, Kawasaki-shi (JP); Yasuhiko TABATA, Uji-shi (JP)

(21) Appl. No.: 16/123,796

(22) Filed: Sep. 6, 2018

(30) Foreign Application Priority Data
Sep. 28, 2017 (JP) 2017-187523

(51) Int. Cl. C12M 1/12; C12N 5/0775; G03F 7/038; G03F 7/16; G03F 7/20

(52) U.S. Cl. CPC C12M 25/14; C12N 5/0663; G03F 7/038; C12N 2533/30; G03F 7/2002; C12N 2539/00; G03F 7/16

(57) ABSTRACT

A cell culture base material for trait induction control of mesenchymal stem cells including an uneven pattern on a surface to which cells adhere and of which the width of the unevenness is greater than or equal to 50 nm and less than 1,000 nm. A trait control method of mesenchymal stem cells includes culturing mesenchymal stem cells on the cell culture base material for trait induction control of mesenchymal stem cells.

### BACKGROUND OF THE INVENTION

### Technical Field

The present invention relates to a cell culture base material for trait induction control of mesenchymal stem cells and a trait control method thereof. Priority is claimed on Japanese Patent Application No. 2017-187523, filed in Japan on Sep. 28, 2017, the contents of which are incorporated herein by reference.

### Background Art

Cell therapy is a therapeutic method of treating a disease (in particular, autoimmune disease) using one's own cells or another person's cells. Examples of the types of cells to be administered include a T cell or a mesenchymal stem cell (MSC). When T cells are used, autotransplantation is performed. When MSCs are used, allotransplantation is performed in most cases as well as autotransplantation. For this reason, MSCs are most industrialized among cells used in cell therapy. In addition, in recent years, it has become clear that a differentiation ability of MSC does not contribute to the therapeutic effect, but a secretory ability of an anti-inflammatory factor contributes thereto. The anti-inflammatory property is being evaluated.

In addition, in recent years, FUJIFILM Corporation has succeeded in greatly enhancing an anti-inflammatory action of MSC by culturing an extracellular matrix and MSCs in combination, the extracellular matrix being independently developed by FUJIFILM Corporation (for example, refer to Non-Patent Document 1).

### CITATION LIST

### Non-Patent Document

[Non-Patent Document 1] FUJIFILM Corporation (president: Kenji Sukeno) “Innovative Research Result in Regenerative Medical Field, Success in significantly Enhancing Anti-Inflammatory Action of Mesenchymal Stem Cells—Improvement of Treatment Effect Using Extracellular Matrix “Cell Nest” suitable for cell culture ˜”, “online”, Mar. 8, 2017, homepage of FUJIFILM Corporation, news release, “search on Aug. 1, 2017”, Internet <URL: http://www.fujifilm.co.jp/corporate/news/articleffnr_1162.html>

### SUMMARY OF THE INVENTION

### Technical Problem

According to the method disclosed in Non-Patent Document 1, it is necessary to induce an anti-inflammatory factor using an inflammatory factor and to use a special extracellular matrix. Therefore, a method for more simply controlling an anti-inflammatory action of MSC is required.

The present invention has been made from the viewpoint of the above-described circumstances and provides a cell culture base material and a trait control method capable of simply controlling trait induction of MSCs in a highly efficient manner.

### Solution to Problem

That is, the present invention includes the following aspects.

A cell culture base material for trait induction control of mesenchymal stem cells according to a first aspect of the present invention includes an uneven pattern on a surface to which cells adhere and of which the width of the unevenness is greater than or equal to 50 nm and less than 1,000 nm.

The uneven pattern may have a lattice shape, a radial shape, a shape in which polygons are continuously formed on a plane, a labyrinth shape, a linear shape, or a dot shape.

A trait control method of mesenchymal stem cells according to a second aspect of the present invention is a method for culturing mesenchymal stem cells on a cell culture base material for trait induction control of mesenchymal stem cells according to the above-described first aspect.

The mesenchymal stem cells may secrete one or more anti-inflammatory factors.

### Advantageous Effects of Invention

According to the cell culture base material of the above-described aspect, it is possible to simply control trait induction of mesenchymal stem cells in a highly efficient manner. According to the trait control method of mesenchymal stem cells of the above-described aspect, it is possible to simply obtain induced mesenchymal stem cells so as to have a specific trait in a highly efficient manner.

### BRIEF DESCRIPTION OF THE DRAWINGS

FIG. 1A is an enlarged plan view and an enlarged front view of a cell culture base material for trait induction control of mesenchymal stem cells (pattern: linear shape) according to an embodiment of the present invention.

FIG. 1B is an enlarged plan view and an enlarged front view of a cell culture base material for trait induction control of mesenchymal stem cells (pattern: dot shape) according to an embodiment of the present invention.

FIG. 2A is a graph showing the number of cells of human bone marrow-derived mesenchymal stem cells (hMSCs-BM) in Example 1.

FIG. 2B is a graph showing the number of cells of human adipose tissue-derived mesenchymal stem cells (hMSCs-AT) in Example 1.

FIG. 3A is a graph showing a protein concentration in a culture supernatant of hMSCs-BM in Example 1.

FIG. 3B is a graph showing a protein concentration in a culture supernatant of hMSCs-AT in Example 1.

FIG. 4 is a graph showing a concentration of prostaglandin E2 (PGE2) in a culture supernatant of hMSCs-BM in Example 1.

### DETAILED DESCRIPTION OF THE INVENTION

Cell Culture Base Material for Trait Induction Control of Mesenchymal Stem Cells

A cell culture base material for trait induction control of mesenchymal stem cells according to an embodiment of the present invention includes an uneven pattern on a surface to which cells adhere and of which the width of the unevenness is greater than or equal to 50 nm and less than 1,000 nm.

By culturing mesenchymal stem cells on an uneven nano pattern of the cell culture base material of the present embodiment as shown in an example to be described below, it is possible to control trait induction of the mesenchymal stem cells without using a trait induction factor such as an inflammatory factor since the uneven shape stimulates the surfaces of cells.

The “trait” referred to herein may be any trait possessed by mesenchymal stem cells, and examples thereof include a cell proliferation ability, a cell differentiation ability, an undifferentiation-maintaining ability, an inflammatory property, an anti-inflammatory property, anti-fibrosis, and a migration ability to a target site. In addition, the “trait induction” means to induce mesenchymal stem cells to have the above exemplified traits.

The mesenchymal stem cells used in the cell culture base material of the present embodiment are cells having an ability to differentiate into cells, such as osteoblasts, adipocytes, myocytes, and chondrocytes, which belong to the mesenchymal system, and can be cells constituting bones, blood vessels, or cardiac muscles.

Examples of the mesenchymal stem cells include bone marrow mesenchymal stem cells, adipose tissue-derived mesenchymal stem cells, synovial tissue-derived mesenchymal stem cells, dental pulp-derived mesenchymal stem cells, tooth germ-derived mesenchymal stem cells, auricular perichondrium-derived mesenchymal stem cells, peripheral blood-derived mesenchymal stem cells, umbilical cord blood-derived mesenchymal stem cells, ligament-derived mesenchymal stem cells, tendon-derived mesenchymal stem cells, ES cell-derived mesenchymal stem cells, and iPS cell-derived mesenchymal stem cells.

In addition, the animal species from which the mesenchymal stem cells are derived is not particularly limited, and may be an invertebrate animal or a vertebrate animal. Examples of the invertebrate animal include crab, shellfish, jellyfish, and shrimp.

Examples of the vertebrate animal include mammals, birds, reptiles, amphibians, and fish.

Examples of the mammals include humans, monkeys, dogs, cats, pigs, sheep, goats, cows, horses, rabbits, guinea pigs, rats, and mice.

Examples of the birds include chickens, quails, ducks, geese, ostriches, and guinea fowl.

Examples of the reptiles include crocodiles, turtles, and lizards.

Examples of the amphibians include frogs, and newts.

Examples of the fishes include tilapia, tai, flounders, sharks, and salmons.

The cell culture base material of the present embodiment has an uneven pattern on a substrate. In addition, the base material is not particularly limited as long as it is not deformed during culturing cells or by subjecting the base material to pretreatment such as sterilization treatment. The entirety of the base material may be made of the same material, or may be made of materials, an uneven pattern and a substrate for supporting the uneven pattern, which are different from each other.

Examples of forms of the base material include a petri dish and a multi-well plate in which any number of wells is disposed. Examples of the number of wells include 6, 12, 24, 96, 384, and 1,536 per plate.

Substrate

In a case where the base material is made of materials, an uneven pattern and a substrate for supporting the uneven pattern, which are different from each other, the material of the substrate is not particularly limited as long as it is used for cell culture. More specific examples of the material of the substrate include glass, polyethylene terephthalate, polycarbonate, cycloolefin polymer, polydimethylsiloxane, and polystyrene. When using these materials, there is a small number of autofluorescent substances, and it is possible to observe cultured cells with a fluorescence microscope.

Uneven Pattern

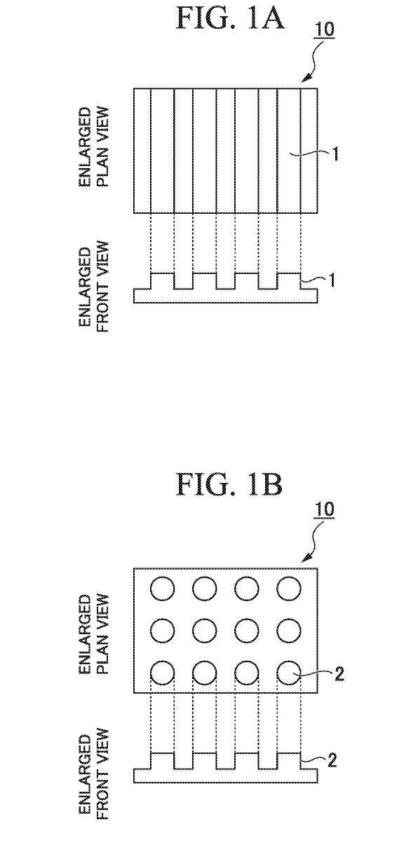

Examples of the uneven pattern include a lattice shape, a radial shape, a shape (for example, honeycomb structure shape) in which polygons are continuously formed on a plane, a labyrinth shape, a linear shape, or a dot shape. FIGS. 1A and 1B are enlarged plan views and enlarged front views of a cell culture base material for trait induction control of mesenchymal stem cells according to an embodiment of the present invention. FIG. 1A shows a case where the uneven pattern has a linear shape. FIG. 1B shows a case where the uneven pattern has a dot shape. In the enlarged front view of FIG. 1A, the shape of a projection portion is a rectangular parallelepiped line shape, but it is not limited thereto, and the shape of the projection portion may be a rectangular column shape (including a rectangular parallelepiped and a cube in a rectangular column), a truncated pyramidal shape, a semicircular column shape (including a semi-elliptical column in a semicircular column), a truncated conical shape (including an elliptical frustum and a biconical truncated cone in a truncated cone), or the like.

In addition, FIG. 1B shows a dot shape in which the transverse section of the projection portion has a circular shape and the longitudinal section thereof has a rectangular shape (that is, the shape of the projection portion is cylindrical). The transverse section or the longitudinal section of the projection portion in the case where the uneven pattern has a dot shape is not limited thereto, and may be polygonal shapes such as a triangle and a quadrangle, a circular shape (including a substantially circular shape, an elliptical shape, a substantially elliptical shape, a semicircular shape, and a fan shape), a trapezoidal shape, and a corrugated shape. That is, examples of the shape of the projection portion in the case where the uneven pattern has a dot shape include a rectangular column shape (including a rectangular parallelepiped and a cube in a rectangular column), a truncated pyramidal shape (including a truncated bipyramid in a truncated pyramid), a circular column shape (including an elliptic column, a semicircular column, a semi-elliptical column, and a sectoral column in a circular column), and a truncated conical shape (including an elliptical frustum and a biconical truncated cone in a truncated cone), or the like, but it is not limited thereto.

The width of the concave portion and the projection portion are preferably 50 nm to 1,000 nm, more preferably 50 nm to 500 nm, and still more preferably 100 nm to 300 nm. By allowing the widths to be within the above-described ranges, it is possible to control trait induction of the mesenchymal stem cells without using a trait induction factor such as an inflammatory factor since the concave portion and the projection portion stimulate the surfaces of mesenchymal stem cells. In a case where the shape of the projection portion is a circular column shape or a truncated conical shape, the width of the projection portion indicates the diameter of the upper surface of the projection portion.

The distance to the surface of the projection portion of the uneven pattern is preferably 10 nm to 100 μm. In a case where the distance to the surface of the projection portion is within the above range, autofluorescence of the substrate is easily suppressed. Therefore, when the substrate having the distance within the above range is used, it is easy to observe the cultured cells by the fluorescence microscope.

Method for Forming Uneven Pattern

The method for forming an uneven pattern is not particularly limited. Examples of the method of forming the uneven pattern include a photolithography method in which a photosensitive composition layer formed on a surface of a substrate for supporting an uneven pattern is selectively exposed, and thereafter a portion corresponding to a concave portion is removed from the exposed photosensitive composition layer with a developing solution, an imprinting method of curing an imprint material after pressing a pressing mold having a pattern of unevenness on a layer of the imprint material formed on a base plate surface, a method in which a mask for covering a portion corresponding to a projection portion is provided on a base plate surface, and thereafter a concave portion is formed on the base plate surface by a chemical treatment such as etching, a method of grinding a base plate surface by sand blasting or various machine tools, a method of attaching a material constituting a projection portion of a pattern having a predetermined shape to a base plate surface, and the like. For the photolithography method and the imprinting method, a photosensitive resin composition used for various purposes in the related art and a photosensitive spin-on-glass (SOG) material can be used without particular limitation.

Photosensitive Resin Composition

Examples of the photosensitive resin composition used for forming the uneven pattern include a photosensitive resin composition containing a resin component, a cationic polymerization initiator, and a solvent, and the like. The photosensitive resin composition may be any of a positive type and a negative type.

The resin component is not particularly limited as long as the resin component can be used for cell culture, for example. Among these, a polymer of a compound having an ethylenic unsaturated bond is preferable. Examples of the polymerizable functional group contained in the compound having an ethylenic unsaturated bond include a (meth)acryloyl group, a vinyl group, an allyl group, and the like. As the compound having the ethylenic unsaturated bond, for example, a monofunctional, a difunctional, or a trifunctional or higher polyfunctional, (meth)acrylate compound, (meth)acrylamide compound, vinyl compound, allyl compound, or the like can be used. These compounds having the ethylenic unsaturated bond can be used alone or in a combination of two or more thereof.

Examples of the polyfunctional compound having the ethylenic unsaturated bond include trifunctional or higher acrylates such as trimethylolpropane tri(meth)acrylate, pentaerythritol tetra(meth)acrylate, ethylene oxide modified pentaerythritol tetra(meth)acrylate, propylene oxide modified pentaerythritol tetra(meth)acrylate, dipentaerythritol penta(meth)acrylate, dipentaerythritol hexa(meth)acrylate, and the like; a polyfunctional urethane (meth)acrylate obtained by reacting a polyisocyanate compound and a hydroxy group-containing (meth)acrylate monomer; and a condensate of polyhydric alcohol and N-methylol(meth)acrylamide, and the like. These polyfunctional compounds can be used alone or in a combination of two or more thereof.

Examples of the difunctional compound having an ethylenically unsaturated bond include polyethylene glycol di(meth)acrylate, polypropylene glycol di(meth)acrylate, polyethylene polypropylene glycol di(meth)acrylate, ethylene glycol di(meth)acrylate, propylene glycol di(meth)acrylate, polyethylene polytrimethylolpropane di(meth)acrylate, 2-(meth)acryloyloxy-2-hydroxypropyl phthalate, 2-(meth)acroyloxyethyl-2-hydroxyethyl phthalate, a compound obtained by reacting α,β-unsaturated carboxylic acid with a glycidyl group-containing compound, an urethane monomer, γ-chloro-β-hydroxypropyl-β′-(meth)acryloyloxyethyl-o-phthalate, β-hydroxyethyl-β′-(meth)acryloyloxyethyl-o-phthalate, β-hydroxypropyl-β′-(meth)acryloyloxyethyl-o-phthalate, and the like.

Examples of the above-described compound obtained by reacting α,β-unsaturated carboxylic acid with a glycidyl group-containing compound include triglycerol di(meth)acrylate. Examples of the above-described urethane monomer include addition reaction products of a (meth)acrylic monomer having a hydroxyl group at the β position and (meth)acryl monomer having a hydroxyl group at the β-position with isophorone diisocyanate, 2,6-toluene diisocyanate, 2,4-toluene diisocyanate, and 1,6-hexamethylene diisocyanate, EO modified urethane di(meth)acrylate, EO/PO modified urethane di(meth)acrylate, and the like.

Examples of the monofunctional compound having an ethylenically unsaturated bond include (meth)acrylic acid esters, (meth)acrylamides, allyl compounds, vinyl ethers, vinyl esters, styrenes, and the like. These compounds can be used alone or in combination of two or more thereof.

Examples of the (meth)acrylic acid esters include methyl (meth)acrylate, ethyl (meth)acrylate, propyl (meth)acrylate, amyl (meth)acrylate, t-octyl (meth)acrylate, chloroethyl (meth)acrylate, 2,2-dimethyl hydroxypropyl (meth)acrylate, 2-hydroxyethyl (meth)acrylate, trimethylolpropane mono(meth)acrylate, benzyl (meth)acrylate, furfuryl (meth)acrylate, phenyl (meth)acrylate, (meth)acrylate of an EO adduct of phenol, (meth)acrylate of a PO adduct of phenol, (meth)acrylate of an EO/PO co-adduct of phenol, ethylene glycol mono(meth)acrylate, diethylene glycol mono(meth)acrylate, triethylene glycol mono(meth)acrylate, polyethylene glycol mono(meth)acrylate, 2-methoxyethyl (meth)acrylate, diethylene glycol monomethyl ether mono(meth)acrylate, triethylene glycol monomethyl ether mono(meth)acrylate, polyethylene glycol monoethyl ether mono(meth)acrylate, propylene glycol mono(meth)acrylate, dipropylene glycol mono(meth)acrylate, tripropylene glycol mono(meth)acrylate, polypropylene glycol mono(meth)acrylate, propylene glycol monomethyl ether mono(meth)acrylate, dipropylene glycol monomethyl ether mono(meth)acrylate, tripropylene glycol monomethyl ether mono(meth)acrylate, polypropylene glycol monomethyl ether mono(meth)acrylate, mono(meth)acrylate of an EO/PO copolymer, monomethyl ether mono(meth)acrylate of an EO/PO copolymer, and the like.

Examples of the (meth)acrylamides include (meth)acrylamide, N-alkyl (meth)acrylamide, N-allyl (meth)acrylamide, N,N-dialkyl (meth)acrylamide, N,N-allyl (meth)acrylamide, N-methyl-N-phenyl (meth)acrylamide, N-hydroxyethyl-N-methyl (meth)acrylamide, and the like.

Examples of the vinyl ethers include alkyl vinyl ethers such as hexyl vinyl ether, octyl vinyl ether, decyl vinyl ether, ethylhexyl vinyl ether, methoxyethyl vinyl ether, ethoxyethyl vinyl ether, chloroethyl vinyl ether, 1-methyl-2,2-dimethylpropyl vinyl ether, 2-ethyl butyl vinyl ether, hydroxyethyl vinyl ether, diethylene glycol vinyl ether, dimethylaminoethyl vinyl ether, diethylaminoethyl vinyl ether, butylaminoethyl vinyl ether, benzyl vinyl ether, and tetrahydrofurfuryl vinyl ether; and vinyl allyl ethers such as vinyl phenyl ether, vinyl tolyl ether, vinyl chlorophenyl ether, vinyl-2,4-dichlorophenyl ether, vinyl naphthyl ether, vinyl anthranyl ether, and the like.

Examples of the vinyl esters include vinyl butyrate, vinyl isobutyrate, vinyl trimethyl acetate, vinyl diethyl acetate, vinyl valate, vinyl caproate, vinyl chloroacetate, vinyl dichloroacetate, vinyl methoxy acetate, vinyl butoxy acetate, vinyl phenyl acetate, vinyl acetoacetate, vinyl lactate, vinyl-β-phenyl butyrate, vinyl benzoate, vinyl salicylate, vinyl chlorobenzoate, vinyl tetrachlorobenzoate, vinyl naphthoate, and the like.

Examples of the styrenes include styrene; alkylstyrenes such as methylstyrene, dimethylstyrene, trimethylstyrene, ethylstyrene, diethylstyrene, isopropylstyrene, butylstyrene, hexylstyrene, cyclohexylstyrene, decylstyrene, benzylstyrene, chloromethylstyrene, trifluoromethylstyrene, ethoxymethylstyrene and acetoxymethylstyrene; alkoxystyrenes such as methoxystyrene, 4-methoxy-3-methylstyrene, and dimethoxystyrene; halostyrenes such as chlorostyrene, dichlorostyrene, trichlorostyrene, tetrachlorostyrene, pentachlorostyrene, bromostyrene, dibromostyrene, iodostyrene, fluorostyrene, trifluorostyrene, 2-bromo-4-trifluoromethylstyrene, 4-fluoro-3-trifluoromethylstyrene, and the like.

The cationic polymerization initiator is a compound which generates a cation by being irradiated with radiation such as ultraviolet rays, far ultraviolet rays, excimer lasers such as KrF and ArF, X-rays, and electron beams, and the cation can become a polymerization initiator.

It is possible to use, for example, an onium salt type cationic polymerization initiator such as an iodonium salt or a sulfonium salt as the cationic polymerization initiator. An anion, as a counter ion of an onium ion, which constitutes the onium salt type cationic polymerization initiator is preferably a fluorinated alkyl fluorophosphoric acid anion, a hexafluorophosphoric acid anion, or a hexafluoroantimonic acid anion (SbF₆—).

A solvent to be contained in the photosensitive resin composition is not particularly limited as long as it is possible to prepare a homogeneous photosensitive resin composition and the effect of exposure is not inhibited. The boiling point of the solvent is preferably 50° C. to 200° C.

Specific examples of the solvent include aliphatic hydrocarbons such as hexane, heptane, octane, decane, and cyclohexane; alcohols such as methanol, ethanol, 1-propanol, 2-propanol, and 1-butanol; and acetone, methyl ethyl ketone, methyl isobutyl ketone, 2-heptanone, ethyl lactate, propylene glycol monomethyl ether acetate, propylene glycol monomethyl ether, ethyl acetate, butyl acetate, ethylene glycol dimethyl ether, propylene glycol dimethyl ether, methyl cellosolve, ethyl cellosolve, dibutyl ether, methyl-3-methoxypropionate, propylene glycol monopropyl ether, butyl cellosolve, diethylene glycol diethyl ether, hexylene glycol, cyclohexanone, propylene glycol monomethyl ether acetate, ethyl pyruvate, ethyl cellosolve acetate, and the like. These may be used alone or in combination of two or more thereof.

In addition to the resin component, the cationic polymerization initiator, and the solvent, the photosensitive resin composition may contain various additives used in the photosensitive resin composition in the related art. Examples of such additives include additional resins, sensitizers, plasticizers, stabilizers, colorants, coupling agents, leveling agents, and the like.

The photosensitive resin composition can be prepared by mixing (dispersing and kneading) each of the above-described components with a stirrer such as a three-roll mill, a ball mill, and a sand mill, and filtering the mixture with a filter such as a 5 μm membrane filter if required.

Method for Producing Base Material

The method for producing a base material is not particularly limited as long as it is a method capable of forming a base material having a desired pattern by exposing and curing the above-described photosensitive resin composition. Examples of the method for producing a cell culture base material include a method including a step of coating a substrate with a photosensitive resin composition to form a coating film and a step of exposing and curing the coating film on the substrate. The above-described method for producing a base material may include a step of exposing and curing a coating film on a substrate, and then, peeling the exposed coating film from the substrate, if required.

In the coating step, the substrate coated with the photosensitive resin composition is not particularly limited as long as it does not cause deformation or deterioration in the process of producing a base material. Examples of the material of the substrate include the same materials as those described above “substrate”.

Examples of a method for forming an uneven pattern include the same methods as those described above “a method for forming an uneven pattern”.

The method for forming a coating film on a substrate is not particularly limited, and examples thereof include a method for adding a predetermined amount of a photosensitive resin composition onto a substrate, a method of using a contact transfer type coating device such as a roll coater, a reverse coater, or a bar coater, a spinner (rotary type coating device), and a non-contact type coating device such as a curtain flow coater.

After the coating film is formed, the substrate provided with the coating film may be placed under a decompressed condition to deaerate the coating film.

In the exposure step, the method for exposing the coating film is not particularly limited as long as it is possible to favorably cure the coating film. The “exposure” is a concept including general irradiation of radiation. For the exposure, for example, a light source emitting ultraviolet rays such as a high-pressure mercury vapor lamp, an ultrahigh pressure mercury vapor lamp, a xenon lamp, a carbon arc lamp or the like may be used. The exposure amount at the time of exposing the coating film is appropriately determined in consideration of the composition of the photosensitive resin composition, the film thickness of the coating film, and the like. Typically, the exposure amount at the time of exposing the coating film is preferably 10 to 100,000 mJ/cm² and more preferably 100 to 50,000 mJ/cm².

The method of exposing the coating film is not particularly limited, but the coating film may be first exposed to the atmosphere to partially cure the coating film. In this manner, in the exposure process, it is possible to prevent the photosensitive resin composition from protruding from the base plate, and thereafter to expose the coating film in water. If the coating film is exposed in water without exposure to the atmosphere, the coating film may dissolve in water in some cases. When the coating film is exposed to the atmosphere and thereafter the coating film is exposed in water, radical polymerization inhibition due to oxygen can be reduced and a good cured film can be obtained.

In addition, the exposure process may include exposure of the coating film in a vacuum. When the coating film is exposed to the vacuum, the coating film of the photosensitive resin composition can be cured in a state of being in close contact with the base plate, and a substrate having a desired pattern is easily formed. In addition, in a case where the coating film is exposed to the vacuum, exposure may be performed while applying pressure to the coating film from the upper surface of the substrate. In this case, the coating film of the photosensitive resin composition can be cured in a state of being in close contact with the substrate. When the exposure process includes exposure to the vacuum or exposure to the vacuum while applying pressure, specifically, in a case where a base material is formed by using a mold corresponding to the pattern of unevenness provided in the base material, it is possible to accurately transfer the uneven pattern of the mold to the base material. By exposing the coating film under such conditions, shrinkage upon curing of the photosensitive resin composition is suppressed, so that the uneven pattern of the mold can be accurately transferred to the base material.

As a method of exposing the coating film to the vacuum, for example, a method in which the surface of the coating film is coated with a film such as a PET film, and thereafter the coating film is exposed at least in a state where a space between the film and the coating film is vacuumed can be mentioned. In a case of exposing while applying pressure to the coating film, as a method of applying pressure to the coating film, for example, a method such as negative pressure exposure can be mentioned.

The coating film that is exposed and cured by the method as described above is used as the base material after detaching from the mold if required.

n addition, the exposed and cured coating film may be subjected to a plasma treatment. By subjecting the cured coating film to the plasma treatment, it is possible to form the base material to which the cell is likely to adhere. Plasma used for the plasma treatment is not particularly limited, but examples thereof include O₂ plasma, N₂ plasma, CF₄ plasma, and the like. The timing of the plasma treatment is not particularly limited, and the plasma treatment may be performed at any timing before or after detaching the cured coating film from the substrate.

Furthermore, the base material detached from the mold may be rinsed with a rinsing liquid. When the base material is rinsed with the rinsing liquid, a compound which can cause cytotoxicity such as an unreacted photopolymerizable monomer or photopolymerization initiator can be removed from the surface of the base material. Examples of the rinsing liquid include organic solvents such as propylene glycol-1-methyl ether acetate (PGMEA), isopropyl alcohol (IPA), and acetone, water, and the like.

Trait Control Method of Mesenchymal Stem Cells

A trait control method of mesenchymal stem cells according to a first embodiment of the present invention is a method for culturing mesenchymal stem cells on a cell culture base material for trait induction control of mesenchymal stem cells according to the above-described embodiment.

According to the trait control method of the present embodiment, it is possible to simply obtain induced mesenchymal stem cells so as to have a specific trait in a highly efficient manner.

Culture Step

In the trait control of the present embodiment, mesenchymal stem cells are cultured using the above-described base material. The temperature for culture is preferably 25° C. to 40° C. The culture period of time can be appropriately set in accordance with the types of cells, and is preferably 1 hour to 50 hours. The trait induction of mesenchymal stem cells is promoted using the above-described base material, and the mesenchymal stem cells can be induced so as to simply have a specific trait in a short period of time compared the method in the related art.

A medium to be used may be a basic medium containing components (such as inorganic salts, carbohydrates, hormones, essential amino acids, nonessential amino acids, and vitamins) or the like required for survival and proliferation of cells, and can be appropriately selected in accordance with the types of cells. Examples thereof include Dulbecco's Modified Eagle's Medium (DMEM), Minimum Essential Medium (MEM), RPMI-1640, Basal Medium Eagle (BME), Dulbecco's Modified Eagle's Medium: Nutrient Mixture F-12 (DMEM/F-12), Glasgow Minimum Essential Medium (Glasgow MEM), and the like. In the culture step, a medium may be appropriately replaced with a new medium in accordance with the proliferation rate of cells.

By using the trait control method of the present embodiment, it is possible to improve, for example, a secretory ability of anti-inflammatory or inflammatory cytokine in mesenchymal stem cells. Among these, mesenchymal stem cells preferably secrete one or more anti-inflammatory cytokines. Examples of the anti-inflammatory or inflammatory cytokines include Adiponectin/Acrp 30, Aggrecan, Angiogenin, Angiopoietin-1, Angiopoietin-2, BAFF/BLyS/TNFSF13B, BDNF, CD14, CD30, CD40 ligand, Chitinase 3-like 1, Complement Component C5/C5a, Complement Factor D, C-Reactive Protein/CRP, Cripto-1, Cystatin C, Dkk-1, DPPIV/CD26, EGF, CXCL5/ENA-78, Endoglin/CD105, EMMPRIN, Fas Ligand, FGF basic, KGF/FGF-7, FGF-19, Flt-3 Ligand, G-CSF, GDF-15, GM-CSF, CXCL1/GRO α, Growth Hormone (GH), HGF, ICAM-1/CD54, IFN-γ, IGFBP-2, IGFBP-3, IL-1α/IL-1F1, IL-1β/IL-1F2, IL-2, IL-3, IL-4, IL-5, IL-6, IL-8, IL-10, IL-11, IL-12 p70, IL-13, IL-15, IL-16, IL-17A, IL-18 BPa, IL-19, IL-22, IL-23, IL-24, IL-27, IL-31, IL-32α/β/γ, IL-33, IL-34, CXCL10/IP-10, CXCL11/I-TAC, Kallikrein 3/PSA, Leptin, LIF, Lipocalin-2/NGAL, CCL2/MCP-1, CCL7/MCP-3, M-CSF, MIF, IL-1ra/IL-1F3, CXCL9/MIG, CCL3/CCL4, MIP-1α/β, CCL20/MIP-3α, CCL19/MIP-3β, MMP-9, Myeloperoxidase, Osteopontin (OPN), PDGF-AA, PDGF-AB/BB, Pentraxin-3, CXCL4/PF4, RAGE, CCL5/RANTES, RBP4, Relaxin-2, Resistin, CXCL12/SDF-1α, Serpin E1/PAI-1, SHBG, ST2/IL-1 R4, CCL17/TARC, TFF3, TfR, TGF-α, Thrombospondin-1, TNF-α, uPAR, VEGF, and Vitamin D BP, and are not limited thereto. One kind of these cytokines may be secreted, or two or more kinds thereof may be secreted.

Application

Mesenchymal stem cells which have been induced through the trait control method of the present embodiment so as to have a specific trait can be suitably used for cell therapy on a patient with an autoimmune disease or the like.

### EXAMPLES

Hereinafter, the present invention will be described using an example, but is not limited to the following example.

### Production Example 1

Preparation of Base Material through Transfer from Argon Fluoride (ArF) Pattern

A radical polymerization type negative resist containing a photosensitive resin composition containing an acrylic resin as a main component was used as a photoresist composition. 1 ml of a radical polymerization negative resist was added dropwise to each 0.8 cm×0.8 cm silicon wafer having an uneven pattern (Smooth, P1 to P4, and LS1 to LS7) which is shown in Table 1 and formed using an ArF exposure machine Nikon S308. A coating film was deaerated while being allowed to stand under a decompressed condition of 100 Pa for 30 minutes, and the radical polymerization negative resist was buried in the uneven pattern. Subsequently, the 12 silicon substrates each having the coating film were exposed at an exposure amount of 999 J/m² in atmospheric air using an ultraviolet irradiation device (HMW-532D manufactured by ORC). Subsequently, each of the films which had been cured through exposure was covered with a substrate obtained by coating a PET substrate (manufactured by Mitsubishi Chemical Corporation) with a radical polymerization negative resist with a film thickness of 1 μm so as to come into contact with the radical polymerization negative resist with a film thickness of 1 μm. The coating films and the radical polymerization negative resists with a film thickness of 1 μm were cured by repeating exposure at an exposure amount of 999 J/m² 5 times using the ultraviolet irradiation device (HMW-532D manufactured by ORC) in vacuum. After peeling the molds from the cured coating films, the cured coating films were immersed in propylene glycol-1-methyl ether acetate (PGMEA) for 10 minutes and rinsed. Thereafter, nitrogen gas was blown to the cured coating films to dry the coating films. Subsequently, O₂ plasma treatment was performed on the dried, cured coating film under the conditions of a pressure of 40 Pa, a temperature of 40° C., an output of 50 W, a treatment time of 20 seconds, and an oxygen flow rate of 200 ml/minute using a plasma treatment device (TCA-3822 manufactured by TOKYO OHKA KOGYO CO., LTD.) to obtain base materials (Smooth, P1 to P4, and LS1 to LS7).

TABLE 1

| | Smooth | P1 | P2 | P3 | P4 | LS1 | LS2 | LS3 | LS4 | LS5 | LS6 | LS7 |
|---|---|---|---|---|---|---|---|---|---|---|---|---|
| Type of Substrate | PET | | | | | | | | | | | |
| Photosensitive | Acrylic resin (radical negative resist) | | | | | | | | | | | |
| resin composition | | | | | | | | | | | | |
| Pattern | — | pillar | | | | Line and Space | | | | | | |
| Width [nm] | 0 | 100 | 200 | 300 | 500 | 75 | 150 | 200 | 250 | 300 | 500 | 1,000 |

In Table 1, “Smooth” represents a flat substrate used as a control, on which an uneven pattern is not formed. In addition, “Width” represents the width of the projection portion.

### Example 1

Trait Induction of Human Bone Marrow-Derived Mesenchymal Stem Cells (hMSCs-BM) and Human Adipose Tissue-Derived Mesenchymal Stem Cells (hMSCs-AT)

(1) Preparation of Cells

Human bone marrow-derived mesenchymal stem cells (hMSCs-BM) and human adipose tissue-derived mesenchymal stem cells (hMSCs-AT) were purchased from Lonza Group Ltd. and used. The cells were cultured in advance.

(2) Trait Induction of Cells

Subsequently, the hMSCs-BM (2.5×10³ cells) and hMSCs-AT (2.0×103 cells) prepared in (1) were seeded in the base materials (Smooth, P1 to P4, and LS1 to LS7) obtained in Production Example 1 or a 24-well plate (made of polystyrene), and cultured overnight. Subsequently, a medium (containing 100 ng/mL of LPS, containing 10 ng/mL of TNFα, or containing no LPS and TNFα) was replaced. Each cell and each culture supernatant were collected 48 hours after the replacement. The base materials (Smooth, P1 to P4, and LS1 to LS7) obtained in Production Example 1 were cultured using a medium containing no LPS and TNFα.

(3) Measurement of Number of Cells

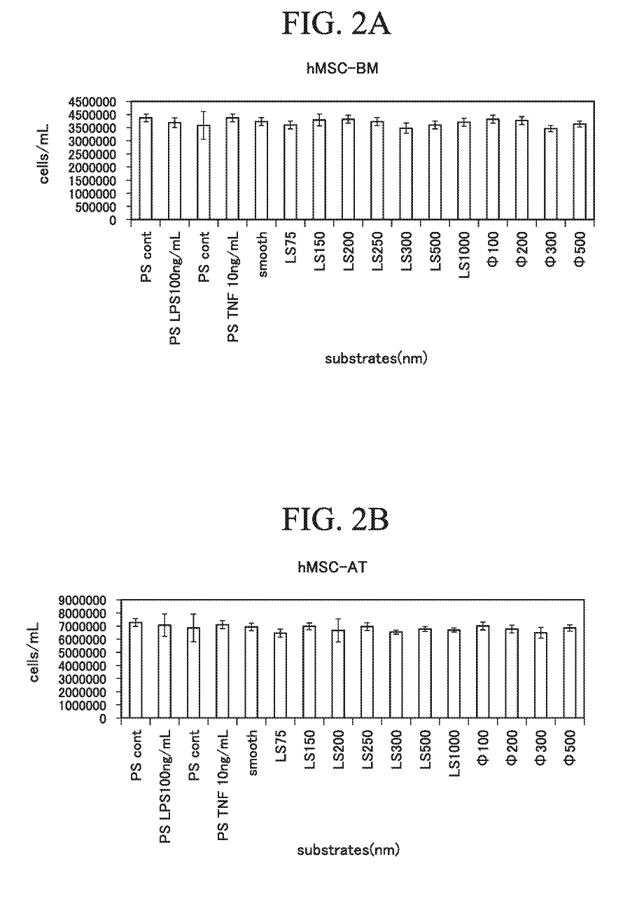

Subsequently, the number of cells of each of the collected MSCs was measured. The results are shown in FIG. 2A (hMSC-BM) and FIG. 2B (hMSC-AT). In FIGS. 2A and 2B, “PS cont” refers to one obtained by culturing hMSCs-BM or hMSCs-AT in a 24-well plate (made of polystyrene). In addition, “PS LPS 100 ng/mL” refers to one obtained by culturing hMSCs-BM or hMSCs-AT with 100 ng/mL of a LPS-containing medium in a 24-well plate (made of polystyrene). In addition, “PS TNF 10 ng/mL” refers to one obtained by culturing hMSCs-BM or hMSCs-AT with 10 ng/mL of a TNFα-containing medium in a 24-well plate (made of polystyrene). In addition, “LS75” to “LS1000” refer to ones obtained by culturing hMSCs-BM or hMSCs-AT on base materials LS1 to LS7. In addition, “ϕ100” to “ϕ500” refer to ones obtained by culturing hMSCs-BM or hMSCs-AT on base materials P1 to P4.

From FIGS. 2A and 2B, there was no significant change in the number of cells depending on the patterns of the base materials. In addition, there was no change in the number of cells depending on LPS or TNFα treatment. In addition, the cell seeding concentration was lower in hMSCs-AT than in hMSCs-BM, but the number of cells after 48 hours in hMSCs-AT was approximately twice as high as that in hMSCs-BM. From this, it was suggested that the cell proliferation ability was higher in hMSCs-AT.

(4) Measurement of Protein Concentration

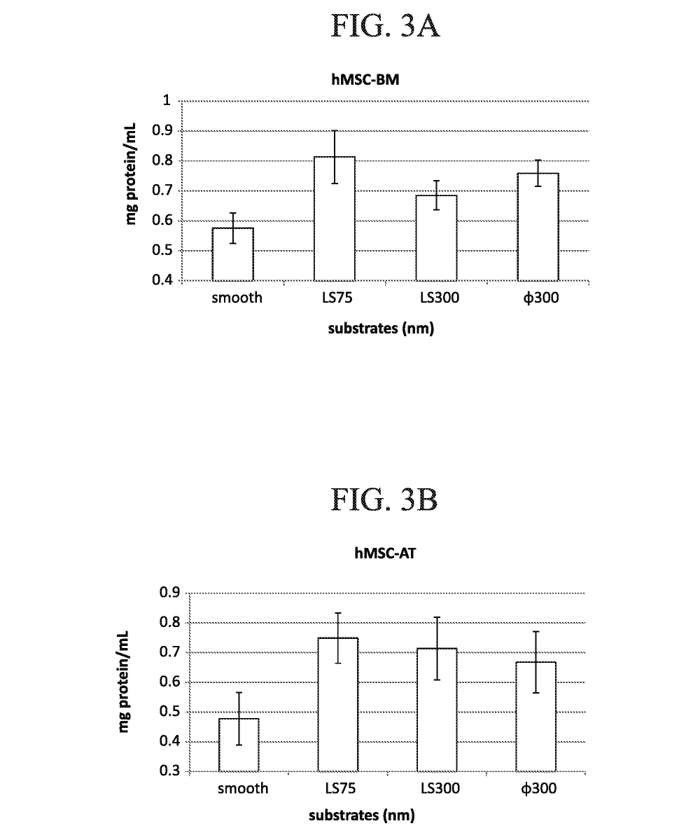

Subsequently, the protein concentration of each of the collected supernatant was measured through a BCA method. The results are shown in FIG. 3A (hMSC-BM) and FIG. 3B (hMSC-AT). Results obtained by culturing hMSCs-BM and hMSCs-AT using the base materials (Smooth, P3 0300), LS1 (LS75), and LS5 (LS300)) obtained in Production Example 1 are shown in FIGS. 3A and 3B.

It became clear from FIGS. 3A and 3B that the protein concentration varies depending on the patterns of the base materials.

(5) Measurement of PGE2 Concentration

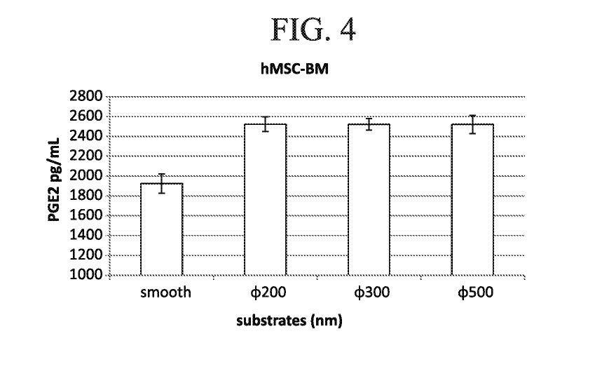

Subsequently, the concentration of prostaglandin E2 (PGE2) was measured using an ELISA kit (manufactured by Enzo Life Sciences). PGE2 is a physiologically functional lipid, and it is known that PGE2 acts on immune cells such as macrophages and strongly suppresses inflammation. The results are shown in FIG. 4. Results obtained by culturing hMSCs-BM using the base materials (Smooth and P2 to P4 (ϕ200 to ϕ500)) obtained in Production Example 1 are shown in FIG. 4.

It became clear from FIG. 4 that the PGE2 concentration was increased depending on the patterns of the base materials.

From the above, it was suggested that the trait induction such as an anti-inflammatory action of MSC can be controlled using the cell culture base material of the present embodiment.

### INDUSTRIAL APPLICABILITY

According to the cell culture base material of the present embodiment, it is possible to simply control trait induction of mesenchymal stem cells in a highly efficient manner. According to the trait control method of mesenchymal stem cells of the present embodiment, it is possible to simply obtain induced mesenchymal stem cells so as to have a specific trait in a highly efficient manner.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1: linear-shaped projection portion, 2: dot-shaped projection portion, 10, 20: base material

What is claimed is:

1. A cell culture base material for trait induction control of mesenchymal stem cells, comprising an uneven pattern on a surface to which cells adhere, and of which the width of the unevenness is greater than or equal to 50 nm and less than 1,000 nm.

2. The cell culture base material for trait induction control of mesenchymal stem cells according to claim 1, wherein the uneven pattern has a lattice shape, a radial shape, a shape in which polygons are continuously formed on a plane, a labyrinth shape, a linear shape, or a dot shape.

3. A trait control method of mesenchymal stem cells, comprising culturing mesenchymal stem cells on the cell culture base material for trait induction control of mesenchymal stem cells according to claim 1.

4. A trait control method of mesenchymal stem cells, comprising culturing mesenchymal stem cells on the cell culture base material for trait induction control of mesenchymal stem cells according to claim 2.

5. The trait control method of mesenchymal stem cells according to claim 3, wherein the mesenchymal stem cells secrete one or more anti-inflammatory factors.

6. The trait control method of mesenchymal stem cells according to claim 4, wherein the mesenchymal stem cells secrete one or more anti-inflammatory factors.
